(19) United States
(12) Patent Application Publication
(10) Pub. No.: US 2026/0016443 A1
(43) Pub. Date: Jan. 15, 2026

(54) GAS SENSOR

(71) Applicant: NGK INSULATORS, LTD., Nagoya-shi (JP)

(72) Inventors: Atsuhiko MASUDA, Nagoya-shi (JP); Daichi ICHIKAWA, Nagoya-shi (JP)

(21) Appl. No.: 19/333,186

(22) Filed: Sep. 18, 2025

Related U.S. Application Data
(63) Continuation of application No. PCT/JP2024/006281, filed on Feb. 21, 2024.

(30) Foreign Application Priority Data
Mar. 30, 2023 (JP) 2023-055669

(51) Int. Cl. G01N 27/41; G01N 27/419; G01N 33/00

(52) U.S. Cl. CPC G01N 27/41; G01N 27/419; G01N 33/004; G01N 33/0073

(57) ABSTRACT

A gas sensor includes a sensor element and a control device, and configured to measure a carbon dioxide concentration in a measurement gas, wherein the control device performs, a first pump cell control processing in which oxygen is pumped out from around a first inner electrode, a second pump cell control processing in which oxygen is pumped to around a second inner electrode by controlling the second pump cell, a third pump cell control processing in which oxygen is pumped to around a third inner electrode by controlling the third pump cell, and the control device derives the carbon dioxide concentration in the measurement gas based on the third pump current flowing through the third pump cell by the third pump cell control processing, while taking into account the second pump current flowing through the second pump cell by the second pump cell control processing.

### CROSS-REFERENCE TO RELATED APPLICATION

This application is a continuation application of PCT/JP2024/006281, filed on Feb. 21, 2024, which claims the benefit of priority of Japanese Application No. 2023-055669, filed on Mar. 30, 2025, the entire contents of which are incorporated herein by reference.

### BACKGROUND OF THE INVENTION

### 1. Field of the Invention

The present invention relates to a gas sensor.

### 2. Description of the Related Art

Hitherto, a gas sensor that detects the concentration of carbon dioxide in a measurement gas such as exhaust gas from an automobile have been known. For example, PTL 1 describes a gas sensor comprising a sensor element including an oxygen-ion-conductive solid electrolyte layer, which specifies the concentrations of water vapor components and carbon dioxide components in the measurement gas. In this gas sensor, the oxygen partial pressure in a first internal cavity of a sensor element is adjusted so that all of the water vapor component and the carbon dioxide component in the measurement-object gas are substantially decomposed in the first internal cavity. The gas sensor then supplies oxygen to a second internal cavity by a first measurement electrochemical pumping cell so that the hydrogen generated by decomposition of the water vapor component is selectively burned in the second internal cavity, and identifies the concentration of the water vapor component present in the measurement-object gas based on the magnitude of a current flowing then. In addition, this gas sensor supplies oxygen to the surface of a second measurement inner electrode by a second measurement electrochemical pumping cell so that the carbon monoxide generated by decomposition of the carbon dioxide component is selectively burned on the surface of the second measurement inner electrode, and identifies the concentration of the carbon dioxide component present in the measurement-object gas based on the magnitude of a current flowing then.

### CITATION LIST

### Patent Literature

PTL 1: JP 5918177 B

### SUMMARY OF THE INVENTION

In such gas sensors, it has been found that even when the concentration of carbon dioxide in the measurement gas is the same, the measurement result of the carbon dioxide concentration by the gas sensor may vary depending on the concentration of the water vapor component (water concentration) in the measurement gas. That is, the measurement accuracy of the carbon dioxide concentration may be degraded due to the presence of water in the measurement gas.

The present invention was made to solve such a problem, and its main object is to suppress a decrease in the measurement accuracy of the carbon dioxide concentration caused by water in the measurement gas.

The present invention employs the following configuration to achieve the above-described main object.

[1] A gas sensor according to the present invention is a gas sensor including a sensor element and a control device, and configured to measure a carbon dioxide concentration in a measurement gas, wherein: the sensor element includes: an element body having an oxygen-ion-conductive solid electrolyte layer and a measurement gas flow path formed therein for introducing and allowing flow of the measurement gas; a first pump cell including a first inner electrode disposed in a first chamber of the measurement gas flow path, and a first outer electrode disposed on the outside of the element body; a second pump cell including a second inner electrode disposed in a second chamber located downstream of the first chamber of the measurement gas flow path, and a second outer electrode disposed on the outside of the element body; a third pump cell including a third inner electrode disposed in a third chamber located downstream of the second chamber of the measurement gas flow path, and a third outer electrode disposed on the outside of the element body; the control device performs: a first pump cell control processing in which oxygen is pumped out from around the first inner electrode to around the first outer electrode by controlling the first pump cell, thereby reducing water and carbon dioxide in the measurement gas in the first chamber; a second pump cell control processing in which oxygen is pumped from around the second outer electrode to around the second inner electrode by controlling the second pump cell, thereby oxidizing hydrogen generated by the reduction of water in the first chamber in the second chamber; a third pump cell control processing in which oxygen is pumped from around the third outer electrode to around the third inner electrode by controlling the third pump cell, thereby oxidizing carbon monoxide generated by the reduction of carbon dioxide in the first chamber in the third chamber; and the control device derives the carbon dioxide concentration in the measurement gas based on the third pump current flowing through the third pump cell by the third pump cell control processing, while taking into account the second pump current flowing through the second pump cell by the second pump cell control processing.

In this gas sensor, the carbon dioxide concentration in the measurement gas is derived based on the third pump current by taking into account the second pump current. Here, the second pump current flowing through the second pump cell by the second pump cell control processing correlates with the water concentration in the measurement gas. Further, the third pump current flowing through the third pump cell by the third pump cell control processing correlates with the carbon dioxide concentration in the measurement gas. However, the third pump current may vary depending on the water concentration in the measurement gas even when the carbon dioxide concentration in the measurement gas is the same. Therefore, by deriving the carbon dioxide concentration in the measurement gas based on the third pump current while taking into account the second pump current, which correlates with the water concentration in the measurement gas, it is possible to suppress a decrease in the measurement accuracy of the carbon dioxide concentration caused by water in the measurement gas.

[2] In the above gas sensor (the gas sensor described in [1]), the control device may derive the carbon dioxide concentration based on a corrected third pump current obtained by correcting the third pump current taking into account the second pump current, or derive the carbon dioxide concentration by correcting a provisional carbon dioxide concentration based on the third pump current taking into account the second pump current.

[3] In the above gas sensor (the gas sensor described in [2]), the control device may derive the corrected third pump current or the carbon dioxide concentration by applying a correction in such a manner that the greater the absolute value of the second pump current, the more the absolute value of the third pump current or the provisional carbon dioxide concentration tends to increase. Here, even when the carbon dioxide concentration in the measurement gas is the same, the absolute value of the third pump current tends to decrease as the water concentration in the measurement gas increases. This is thought to be because the higher the water concentration in the measurement gas, the more insufficient the reduction of carbon dioxide in the first chamber becomes due to the first pump cell control processing. Therefore, by applying a correction in such a manner that the greater the absolute value of the second pump current, which correlates with the water concentration, the more the absolute value of the third pump current or the provisional carbon dioxide concentration tends to increase, it is possible to more reliably suppress a decrease in the measurement accuracy of the carbon dioxide concentration caused by water in the measurement gas.

[4] In the above gas sensor (the gas sensor described in [2]), the control device may derive the corrected third pump current or the carbon dioxide concentration by applying a correction in such a manner that the greater the absolute value of the second pump current, the more the absolute value of the third pump current or the provisional carbon dioxide concentration tends to decrease. Here, in some cases, the absolute value of the third pump current may tend to increase as the water concentration in the measurement gas increases even when the carbon dioxide concentration in the measurement gas is the same. For example, such a tendency may be observed when the amount of measurement gas reaching the first chamber from outside the element body is relatively small, such as in a case where the diffusion resistance from outside the element body to the first chamber in the measurement gas flow path is relatively high. This reason is considered as follows. First, when the amount of measurement gas reaching the first chamber from outside is relatively small, the reduction of carbon dioxide in the first chamber by the first pump cell control processing is less likely to become insufficient. On the other hand, since water (and hydrogen generated by the reduction of water) has a relatively high diffusion rate, water has an effect of promoting the diffusion of carbon dioxide (and carbon monoxide generated by the reduction of carbon dioxide) in the measurement gas. Therefore, when the reduction of carbon dioxide in the first chamber is less likely to become insufficient, the effect of water and hydrogen promoting the diffusion of carbon dioxide and carbon monoxide becomes dominant. As a result, it is considered that the higher the water concentration in the measurement gas, the more the amount of carbon monoxide reaching the third chamber increases, and a tendency appears in which the absolute value of the third pump current increases, even if the carbon dioxide concentration in the measurement gas is the same. Therefore, by applying a correction in such a manner that the greater the absolute value of the second pump current, which correlates with the water concentration, the more the absolute value of the third pump current or the provisional carbon dioxide concentration tends to decrease, it is possible to more reliably suppress a decrease in the measurement accuracy of the carbon dioxide concentration caused by water in the measurement gas.

[5] In the above gas sensor (the gas sensor described in any one of [2] to [4]), the control device may derive a correction value based on the absolute value of the second pump current, and correct the third pump current or the provisional carbon dioxide concentration using the correction value. In this case, the control device may derive the correction value based on both the absolute value of the second pump current and the absolute value of the third pump current, and correct the third pump current using the derived correction value. Alternatively, the control device may derive the correction value based on both the absolute value of the second pump current and the provisional carbon dioxide concentration, and correct the provisional carbon dioxide concentration using the derived correction value.

[6] In the above gas sensor (the gas sensor described in [5]), the correction value may be a multiplication coefficient for the absolute value of the third pump current or the provisional carbon dioxide concentration, and the control device may correct the third pump current or the provisional carbon dioxide concentration by multiplying the absolute value of the third pump current or the provisional carbon dioxide concentration by the correction value.

[7] In the above gas sensor (the gas sensor described in [6]), the control device may correct the third pump current or the provisional carbon dioxide concentration using the same correction value derived based on the absolute value of the second pump current, regardless of the magnitude of the absolute value of the third pump current or the provisional carbon dioxide concentration. When the correction value is the multiplication coefficient, relatively accurate correction of the third pump current or the provisional carbon dioxide concentration can be achieved even using the same correction value based on the absolute value of the second pump current, regardless of the magnitude of the absolute value of the third pump current or the provisional carbon dioxide concentration. Therefore, the correction can be performed more easily compared to the case where the correction value is derived based on not only the absolute value of the second pump current but also the absolute value of the third pump current or the provisional carbon dioxide concentration.

[8] In the above gas sensor (the gas sensor described in any one of [1] to [7]), the control device may derive a water concentration in the measurement gas based on the second pump current. In this manner, this gas sensor can measure not only the carbon dioxide concentration but also the water concentration. In this case, the control device may derive the carbon dioxide concentration based on the third pump current while taking into account the water concentration derived based on the absolute value of the second pump current. For example, a correction taking the water concentration into account may be applied to the third pump current, or a correction taking the water concentration into account may be applied to the provisional carbon dioxide concentration. These embodiments are also included as specific examples of deriving the carbon dioxide concentration based on the third pump current while taking into account the second pump current. For example, the control device may derive the correction value based on the water concentration derived from the absolute value of the second pump current.

[9] In the above gas sensor (the gas sensor described in any one of [1] to [8]), at least two of the first outer electrode, the second outer electrode, and the third outer electrode may be shared electrodes.

[10] In the above gas sensor (the gas sensor described in any one of [1] to [9]), the sensor element may include a reference electrode disposed inside the element body to contact a reference gas, and the control device may control the first pump cell such that a first voltage between the reference electrode and the first inner electrode reaches a first voltage target value in the first pump cell control processing, the second pump cell such that a second voltage between the reference electrode and the second inner electrode reaches a second voltage target value in the second pump cell control processing, and the third pump cell such that a third voltage between the reference electrode and the third inner electrode reaches a third voltage target value in the third pump cell control processing.

### BRIEF DESCRIPTION OF THE DRAWINGS

FIG. 1 is a schematic cross-sectional view schematically showing an example of a configuration of a gas sensor 100.

FIG. 2 is a block diagram showing the electrical connections between a control device 95 and respective cells and other components.

FIG. 3 is a flowchart showing one example of the concentration derivation processing routine.

FIG. 4 is an explanatory diagram showing one example of a water concentration derivation map.

FIG. 5 is an explanatory diagram showing one example of a correction value derivation map.

FIG. 6 is a graph showing a relationship between a water concentration Cw and the absolute value of a pump current Ip2.

FIG. 7 is a graph showing a relationship between the water concentration Cw and the absolute value of the pump current Ip2 when FIG. 6 is normalized.

FIG. 8 is an explanatory diagram showing one example of a carbon dioxide concentration derivation map.

FIG. 9 is a graph showing the relationship between a carbon dioxide concentration Ccd and the absolute value of the pump current Ip2.

FIG. 10 is an explanatory diagram showing another example of a correction value derivation map.

FIG. 11 is a schematic cross-sectional view of a sensor element 201 according to a modification.

### DETAILED DESCRIPTION OF THE INVENTION

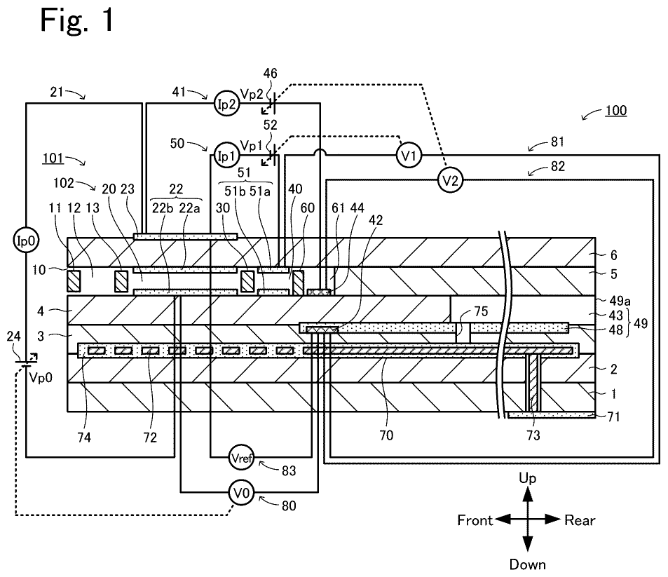

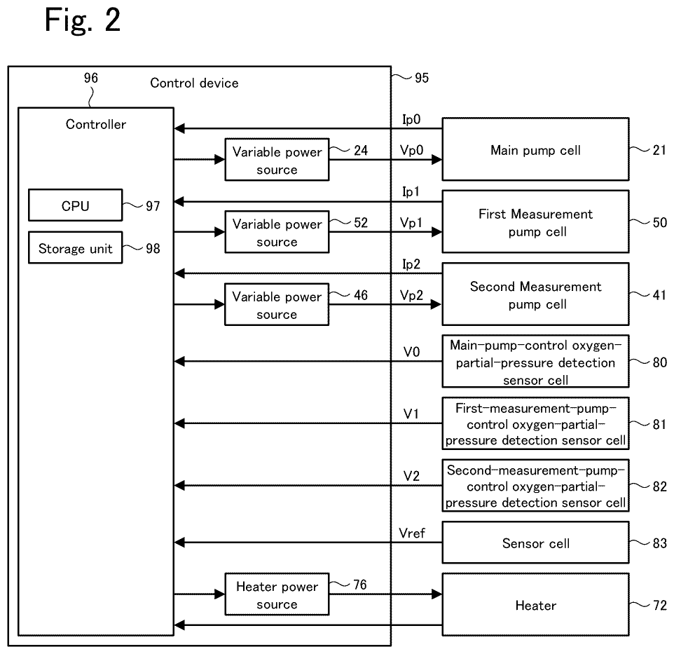

Next, embodiments of the present invention will be described with reference to the drawings. FIG. 1 is a schematic cross-sectional view schematically showing an example of a configuration of a gas sensor 100 according to an embodiment of the present invention. FIG. 2 is a block diagram showing the electrical connections between a control device 95, respective cells and a heater 72. The gas sensor 100 is installed in a pipe, such as an exhaust pipe of an internal combustion engine. The gas sensor 100 detects a concentration of a specific gas in a measurement gas, using exhaust gas from an internal combustion engine as the measurement gas. In the present embodiment, the gas sensor 100 is configured to detect the carbon dioxide concentration and the water concentration as the concentrations of the specific gases. The gas sensor 100 includes: a sensor element 101 with an elongated rectangular parallelepiped element body 102; cells 21, 41, 50, and 80 to 83 within the sensor element 101; a heater section 70 provided inside the sensor element 101; and a control device 95, which includes variable power sources 24, 46, and 52, and a heater power source 76, and controls the overall operation of the gas sensor 100. Note that the longitudinal direction (left-right direction in FIG. 1) of the sensor element 101 is defined as the front-rear direction, the thickness direction (up-down direction in FIG. 1) of the sensor element 101 as the up-down direction, and the width direction (perpendicular to both front-rear direction and up-down direction) of the sensor element 101 as the left-right direction.

The element body 102 is a laminated body in which six layers are stacked in the following order from the bottom in the drawing: a first substrate layer 1, a second substrate layer 2, a third substrate layer 3, a first solid electrolyte layer 4, a spacer layer 5, and a second solid electrolyte layer 6. Each of these layers is composed of an oxygen-ion-conductive solid electrolyte layer, such as zirconia (ZrO₂) or the like. The solid electrolytes forming these six layers are dense and hermetically sealed. The element body 102 is manufactured, for example, by performing predetermined processing and printing of circuit patterns on ceramic green sheets corresponding to the respective layers, laminating the sheets, and then firing the laminated sheets to integrate them into a unified structure.

On the front end side of the sensor element 101 (element body 102), between the lower surface of the second solid electrolyte layer 6 and the upper surface of the first solid electrolyte layer 4, the following components are formed adjacently and connected in sequence: a gas inlet 10; a first diffusion rate-limiting section 11; a buffer space 12; a second diffusion rate-limiting section 13; a first internal cavity 20; a third diffusion rate-limiting section 30; a second internal cavity 40; a fourth diffusion rate-limiting section 60; and a third internal cavity 61.

The gas inlet 10, buffer space 12, first internal cavity 20, second internal cavity 40, and third internal cavity 61 are internal spaces within the sensor element 101, formed by hollowing out portions of the spacer layer 5. These spaces are bounded at the top by the lower surface of the second solid electrolyte layer 6, at the bottom by the upper surface of the first solid electrolyte layer 4, and on the sides by the side surfaces of the spacer layer 5.

The first diffusion rate-limiting section 11, the second diffusion rate-limiting section 13, and the third diffusion rate-limiting section 30 are each provided as two horizontally elongated slits, with openings oriented along the longitudinal direction perpendicular to the plane of the drawing. The fourth diffusion rate-limiting section 60 is provided as a single horizontally elongated slit, with openings oriented along the longitudinal direction perpendicular to the plane of the drawing, formed as a gap with the lower surface of the second solid electrolyte layer 6. The area extending from the gas inlet 10 to the third internal cavity 61 is also referred to as the measurement gas flow path.

The sensor element 101 (element body 102) includes a reference gas introduction portion 49, which introduces a reference gas from outside of the sensor element 101 to a reference electrode 42 when measuring the concentrations of the specific gases. The reference gas introduction portion 49 comprises a reference gas introduction space 43 and a reference gas introduction layer 48. The reference gas introduction space 43 is an inward space formed from the rear end surface of the sensor element 101. The reference gas introduction space 43 is located between the upper surface of the third substrate layer 3 and the lower surface of the spacer layer 5, and is laterally defined by the side surfaces of the first solid electrolyte layer 4. The reference gas introduction space 43 opens to the rear end surface of the sensor element 101, with this opening serving as an inlet portion 49a of the reference gas introduction portion 49. The reference gas is introduced into the reference gas introduction space 43 through the inlet portion 49a. The reference gas introduction portion 49 introduces the reference gas, which has entered through the inlet portion 49a, to the reference electrode 42 while imparting a predetermined diffusion resistance. In the present embodiment, the reference gas is ambient air.

The reference gas introduction layer 48 is provided between the upper surface of the third substrate layer 3 and the lower surface of the first solid electrolyte layer 4. The reference gas introduction layer 48 is a porous body made of a ceramic material such as alumina or the like. A portion of the upper surface of the reference gas introduction layer 48 is exposed within the reference gas introduction space 43. The reference gas introduction layer 48 is formed so as to cover the reference electrode 42. The reference gas introduction layer 48 allows the reference gas to flow from the reference gas introduction space 43 to the reference electrode 42.

The reference electrode 42 is an electrode formed between the upper surface of the third substrate layer 3 and the first solid electrolyte layer 4, and as described above, the reference gas introduction layer 48, which is connected to the reference gas introduction space 43, is provided around the reference electrode 42. Furthermore, as will be explained later, the reference electrode 42 enables the measurement of the oxygen concentration (oxygen partial pressure) in the first internal cavity 20, the second internal cavity 40, and the third internal cavity 61. The reference electrode 42 is formed as a porous cermet electrode (for example, a cermet electrode of Pt and ZrO₂).

In the measurement gas flow path, the gas inlet 10 is a portion that is open to the external space, allowing the measurement gas to be drawn into the sensor element 101 from the external space. The first diffusion rate-limiting section 11 is a part that imparts a predetermined diffusion resistance to the measurement gas introduced through the gas inlet 10. The buffer space 12 is a space provided to guide the measurement gas introduced through the first diffusion rate-limiting section 11 to the second diffusion rate-limiting section 13. The second diffusion rate-limiting section 13 is a portion that imparts a predetermined diffusion resistance to the measurement gas introduced from the buffer space 12 into the first internal cavity 20. When the measurement gas is introduced from outside the sensor element 101 into the first internal cavity 20, the measurement gas that is abruptly drawn into the sensor element 101 through the gas inlet 10 due to pressure fluctuations in the external space (such as exhaust pulsations in the case where the measurement gas is automobile exhaust gas) is not directly introduced into the first internal cavity 20. Instead, after the pressure fluctuations of the measurement gas are attenuated through the first diffusion rate-limiting section 11, the buffer space 12, and the second diffusion rate-limiting section 13, the measurement gas is introduced into the first internal cavity 20. As a result, the pressure fluctuations of the measurement gas introduced into the first internal cavity 20 become almost negligible. The first internal cavity 20 is provided as a space for adjusting the oxygen partial pressure in the measurement gas introduced through the second diffusion rate-limiting section 13. This oxygen partial pressure is adjusted by the operation of a main pump cell 21.

The main pump cell 21 is an electrochemical pump cell, which is constituted by an inner pump electrode 22 with a ceiling electrode portion 22a provided on nearly the entire lower surface of the second solid electrolyte layer 6 facing the first internal cavity 20, an outer pump electrode 23, which is provided in a manner exposed to the outside of the sensor element 101 in a region of the upper surface of the second solid electrolyte layer 6 corresponding to the ceiling electrode portion 22a, and the second solid electrolyte layer 6, the spacer layer 5, and the first solid electrolyte layer 4, which form the current path between these electrodes.

The inner pump electrode 22 is formed so as to extend across the upper and lower solid electrolyte layers, (namely the second solid electrolyte layer 6 and the first solid electrolyte layer 4,) and the spacer layer 5 that provides sidewalls, which together define the first internal cavity 20. Specifically, the ceiling electrode portion 22a is formed on the lower surface of the second solid electrolyte layer 6, which constitutes the ceiling surface of the first internal cavity 20, and a bottom electrode portion 22b is formed on the upper surface of the first solid electrolyte layer 4, which constitutes the bottom surface of the first internal cavity 20. Further, in order to connect the ceiling electrode portion 22a and the bottom electrode portion 22b, side electrode portions (not shown) are formed on the side wall surfaces (inner surfaces) of the spacer layer 5, which constitute both sidewall portions of the first internal cavity 20. The inner pump electrode 22 is disposed in a tunnel-like structure at the region where the side electrode portion is provided.

The inner pump electrode 22 and the outer pump electrode 23 each are formed as a porous cermet electrode (for example, a cermet electrode of Pt and ZrO₂, having an Au content of 1%).

In the main pump cell 21, a desired voltage Vp0 is applied between the inner pump electrode 22 and the outer pump electrode 23, whereby a pump current Ip0 is caused to flow in a positive direction or a negative direction between the inner pump electrode 22 and the outer pump electrode 23. Thus, the oxygen in the first internal cavity 20 can be pumped out to the external space, or the oxygen in the external space can be pumped into the first internal cavity 20.

Further, in order to detect the oxygen concentration (oxygen partial pressure) in the atmosphere within the first internal cavity 20, an electrochemical sensor cell, that is, a main-pump-control oxygen-partial-pressure detection sensor cell 80, is constituted by the inner pump electrode 22, the second solid electrolyte layer 6, the spacer layer 5, the first solid electrolyte layer 4, the third substrate layer 3, and the reference electrode 42.

By measuring an electromotive force (voltage V0) in the main-pump-control oxygen-partial-pressure detection sensor cell 80, the oxygen concentration (oxygen partial pressure) in the first internal cavity 20 can be determined. Furthermore, by feedback-controlling the voltage Vp0 of the variable power source 24 such that the voltage V0 reaches a target value, the pump current Ip0 is controlled, thereby adjusting the oxygen concentration in the first internal cavity 20.

The third diffusion rate-limiting section 30 is a part that imparts a predetermined diffusion resistance to the measurement gas, whose oxygen concentration (oxygen partial pressure) is controlled by the operation of the main pump cell 21 in the first internal cavity 20, and guides the measurement gas into the second internal cavity 40.

The second internal cavity 40 is provided as a space in which the oxygen partial pressure is adjusted by the first measurement pump cell 50 for the measurement gas introduced through the third diffusion rate-limiting section 30, to carry out processing for measuring the water concentration in the measurement gas.

The first measurement pump cell 50 is an electrochemical pump cell, which is constituted by a first measurement electrode 51 with a ceiling electrode portion 51a provided on nearly the entire lower surface of the second solid electrolyte layer 6 facing the second internal cavity 40, the outer pump electrode 23 (not limited to the outer pump electrode 23, but may be any suitable electrode located outside the sensor element 101), the second solid electrolyte layer 6, the spacer layer 5, and the first solid electrolyte layer 4.

The first measurement electrode 51 is disposed within the second internal cavity 40 in a tunnel-like structure similar to that of the inner pump electrode 22 disposed in the first internal cavity 20 described above. Specifically, the ceiling electrode portion 51a is formed on the second solid electrolyte layer 6, which constitutes the ceiling surface of the second internal cavity 40, and a bottom electrode portion 51b is formed on the first solid electrolyte layer 4, which constitutes the bottom surface of the second internal cavity 40. Further, side electrode portions (not shown), which connect the ceiling electrode portion 51a and the bottom electrode portion 51b, are formed on the inner side surfaces of the spacer layer 5, which constitute both sidewall portions of the second internal cavity 40. Thus, the first measurement electrode 51 is formed in a tunnel-like structure. The first measurement electrode 51 is also formed as a porous cermet electrode using the same material as the inner pump electrode 22.

In the first measurement pump cell 50, a desired voltage Vp1 is applied between the first measurement electrode 51 and the outer pump electrode 23. Thus, the oxygen in the atmosphere within the second internal cavity 40 can be pumped out to the external space, or the oxygen can be pumped into the second internal cavity 40 from the external space.

Further, in order to control the oxygen partial pressure in the atmosphere within the second internal cavity 40, an electrochemical sensor cell, that is, an first-measurement-pump-control oxygen-partial-pressure detection sensor cell 81, is constituted by the first measurement electrode 51, the reference electrode 42, the second solid electrolyte layer 6, the spacer layer 5, the first solid electrolyte layer 4, and the third substrate layer 3.

The first measurement pump cell 50 performs pumping via the variable power source 52, which is voltage-controlled based on an electromotive force (voltage V1) detected by the first-measurement-pump-control oxygen-partial-pressure detection sensor cell 81. As a result, the oxygen partial pressure in the atmosphere of the second internal cavity 40 is adjusted by the pump current Ip1 flowing through the first measurement pump cell 50.

The fourth diffusion rate-limiting section 60 is a part that imparts a predetermined diffusion resistance to the measurement gas, whose oxygen concentration (oxygen partial pressure) is controlled by the operation of the first measurement pump cell 50 in the second internal cavity 40, and guides the measurement gas into the third internal cavity 61.

The third internal cavity 61 is provided as a space in which the oxygen partial pressure is adjusted by the second measurement pump cell 41 for the measurement gas introduced through the fourth diffusion rate-limiting section 60, to carry out processing for measuring the carbon dioxide concentration in the measurement gas.

The second measurement pump cell 41 is an electrochemical pump cell, which is constituted by a second measurement electrode 44 provided on the upper surface of the first solid electrolyte layer 4 facing the third internal cavity 61, the outer pump electrode 23, the second solid electrolyte layer 6, the spacer layer 5, and the first solid electrolyte layer 4. The second measurement electrode 44 is also formed as a porous cermet electrode using the same material as the inner pump electrode 22.

In the second measurement pump cell 41, a desired voltage Vp2 is applied between the second measurement electrode 44 and the outer pump electrode 23. Thus, the oxygen in the atmosphere within the third internal cavity 61 can be pumped out to the external space, or the oxygen can be pumped into the third internal cavity 61 from the external space.

Further, in order to detect the oxygen partial pressure around the second measurement electrode 44, an electrochemical sensor cell, that is, a second-measurement-pump-control oxygen-partial-pressure detection sensor cell 82, is formed by the first solid electrolyte layer 4, the third substrate layer 3, the second measurement electrode 44, and the reference electrode 42.

The variable power source 46 is controlled based on the electromotive force (voltage V2) detected by the second-measurement-pump-control oxygen-partial-pressure detection sensor cell 82, and the voltage Vp2 of the variable power source 46 is applied to the second measurement pump cell 41. As a result, the oxygen partial pressure in the atmosphere of the third internal cavity 61 is adjusted by the pump current Ip2 flowing through the second measurement pump cell 41.

Furthermore, an electrochemical sensor cell 83 is formed from the second solid electrolyte layer 6, the spacer layer 5, the first solid electrolyte layer 4, the third substrate layer 3, the outer pump electrode 23, and the reference electrode 42, and the oxygen partial pressure in the measurement gas outside the sensor can be detected based on the electromotive force (voltage Vref) obtained by this sensor cell 83.

The sensor element 101 includes a heater section 70 that performs temperature regulation by heating and maintaining the temperature of the sensor element 101, in order to enhance the oxygen-ion-conductivity of the solid electrolyte. The heater section 70 includes a heater connector electrode 71, a heater 72, a through hole 73, a heater insulating layer 74, and a pressure relief hole 75.

The heater connector electrode 71 is an electrode formed in such a manner as to be in contact with the lower surface of the first substrate layer 1. By connecting the heater connector electrode 71 to a heater power source 76 (see FIG. 2), power can be supplied from the heater power source 76 to the heater section 70.

The heater 72 is an electrical resistor formed in such a manner as to be sandwiched between the second substrate layer 2 and the third substrate layer 3 from above and below. The heater 72 is connected to the heater connector electrode 71 via the through hole 73, and generates heat when power is supplied from the heater power source 76 through the heater connector electrode 71, thereby heating and maintaining the temperature of the solid electrolyte forming the sensor element 101.

The heater 72 is also embedded across the entire region from the first internal cavity 20 to the third internal cavity 61, making it possible to adjust the temperature of the entire sensor element 101 to a level at which the solid electrolyte is activated.

The heater insulating layer 74 is an insulating layer formed of an insulator such as alumina and provided on the upper and lower surfaces of the heater 72. The heater insulating layer 74 is formed for the purpose of providing electrical insulation between the second substrate layer 2 and the heater 72, as well as between the third substrate layer 3 and the heater 72.

The pressure relief hole 75 is a portion that penetrates through the third substrate layer 3 and the reference gas introduction layer 48, and is formed so as to communicate with the reference gas introduction space 43. The pressure relief hole 75 is formed for the purpose of relieving an increase in internal pressure caused by a rise in temperature within the heater insulating layer 74.

As shown in FIG. 2, the control device 95 includes the variable power sources 24, 46, and 52 described above, the heater power source 76 also described above, and a control unit 96. The control unit 96 is a microprocessor including a CPU 97 and a storage unit 98. The storage unit 98 is a rewritable non-volatile memory, and is capable of storing, for example, various programs and various kinds of data. The control unit 96 inputs the voltage V0 from the main-pump-control oxygen-partial-pressure detection sensor cell 80, the voltage V1 from the first-measurement-pump-control oxygen-partial-pressure detection sensor cell 81, the voltage V2 from the second-measurement-pump-control oxygen-partial-pressure detection sensor cell 82, the voltage Vref from the sensor cell 83, the pump current Ip0 flowing through the main pump cell 21, the pump current Ip1 flowing through the first measurement pump cell 50, and the pump current Ip2 flowing through the second measurement pump cell 41. In addition, the control unit 96 controls the voltages Vp0, Vp1, and Vp2 output from the variable power sources 24, 46, and 52, respectively, by outputting control signals to the variable power sources 24, 46, and 52. Through this control, the main pump cell 21, the second measurement pump cell 41, and the first measurement pump cell 50 are controlled. The control unit 96 also controls the power supplied from the heater power source 76 to the heater 72 by outputting a control signal to the heater power source 76. The storage unit 98 also stores target values V0*, V1*, and V2*, which will be described later. The CPU 97 of the control unit 96 performs control of the respective cells 21, 41, and 50 with reference to the target values V0*, V1*, and V2*.

The control unit 96 performs a main pump control processing (an example of the first pump cell control processing), which controls the main pump cell 21 to pump out oxygen from around the inner pump electrode 22 to around the outer pump electrode 23. Specifically, the control unit 96 feedback-controls the voltage Vp0 of the variable power source 24 so that the voltage V0 reaches a target value V0*, thereby controlling the main pump cell 21. The target value V0* is set as a value such that the oxygen concentration in the first internal cavity 20 reaches a predetermined low concentration that is sufficiently low to substantially reduce all of the water and carbon dioxide in the measurement gas. By performing this main pump control processing, in the first internal cavity 20, water in the measurement gas is reduced to generate hydrogen and oxygen, and carbon dioxide in the measurement gas is reduced to generate carbon monoxide and oxygen. The generated oxygen is pumped out from around the inner pump electrode 22 to around the outer pump electrode 23 by the pump current Ip0 flowing through the main pump cell 21.

The control unit 96 performs a first measurement pump control processing (an example of the second pump cell control processing), which controls the first measurement pump cell 50 to pump into oxygen from around the outer pump electrode 23 to around the first measurement electrode 51. Specifically, the control unit 96 feedback-controls the voltage Vp1 of the variable power source 52 so that the voltage V1 reaches the target value V1*, thereby controlling the first measurement pump cell 50. The target value V1* is set as a value such that the oxygen concentration in the second internal cavity 40 reaches a predetermined concentration sufficient to substantially oxidize all of hydrogen in the second internal cavity 40. By performing this first measurement pump control processing, in the second internal cavity 40, hydrogen generated by the reduction of water in the first internal cavity 20 is oxidized to generate water again. At this time, the pump current Ip1 flowing through the first measurement pump cell 50 correlates with the amount of oxygen pumped into the second internal cavity 40 to oxidize the hydrogen in the second internal cavity 40, and hence correlates with the amount of water in the measurement gas in the first internal cavity 20, which is the source of the hydrogen in the second internal cavity 40. Therefore, the pump current Ip1 correlates with the water concentration in the measurement gas and the water concentration in the measurement gas can be detected based on the pump current Ip1.

The control unit 96 performs a second measurement pump control processing (an example of the third pump cell control processing), which controls the second measurement pump cell 41 to pump into oxygen from around the outer pump electrode 23 to around the second measurement electrode 44. Specifically, the control unit 96 feedback-controls the voltage Vp2 of the variable power source 46 so that the voltage V2 reaches the target value V2*, thereby controlling the second measurement pump cell 41. The target value V2* is set as a value such that the oxygen concentration in the third internal cavity 61 reaches a predetermined concentration sufficient to substantially oxidize all of carbon monoxide in the third internal cavity 61. By performing this second measurement pump control processing, in the third internal cavity 61, carbon monoxide generated by the reduction of carbon dioxide in the first internal cavity 20 is oxidized to generate carbon dioxide again. At this time, the pump current Ip2 flowing through the second measurement pump cell 41 correlates with the amount of oxygen pumped into the third internal cavity 61 to oxidize the carbon monoxide in the third internal cavity 61, and hence correlates with the amount of carbon dioxide in the measurement gas in the first internal cavity 20, which is the source of the carbon monoxide in the third internal cavity 61. Therefore, the pump current Ip2 correlates with the carbon dioxide concentration in the measurement gas and the carbon dioxide concentration in the measurement gas can be detected based on the pump current Ip2. In the present embodiment, however, the control unit 96 corrects the pump current Ip2 based on the pump current Ip1 to derive a corrected pump current Ip2ad, and derives the carbon dioxide concentration based on the corrected pump current Ip2ad. Details of the correction will be described later.

Both hydrogen and carbon monoxide generated in the first internal cavity 20 reach the second internal cavity 40. However, hydrogen has a faster gas diffusion rate than carbon monoxide and hydrogen binds with oxygen more readily than carbon monoxide does. Therefore, in the second internal cavity 40, hydrogen can be selectively oxidized, among hydrogen and carbon monoxide, by the first measurement pump control processing. Additionally, since hydrogen rarely reaches the third internal cavity 61 downstream of the second internal cavity 40, the second measurement pump control processing can oxidize carbon monoxide.

The control unit 96 performs heater control processing by outputting a control signal to the heater power source 76 such that the temperature of the heater 72 reaches to a target temperature (e.g. 800° C.). Here, the temperature of the heater 72 can be expressed as a linear function of the resistance value of the heater 72. In the heater control processing, the control unit 96 calculates the resistance value of the heater 72, which is a value can be regarded as the temperature of the heater 72 (a value that can be converted into temperature), and feedback-controls the heater power source 76 such that the calculated resistance value reaches to a target resistance value (a value corresponding to the target temperature). The control unit 96 can acquire the voltage of the heater 72 and the current flowing through the heater 72, then calculate the resistance of the heater 72 based on the acquired voltage and current. The control unit 96 may use a three-wire or four-wire method to calculate the resistance, for example. The heater power source 76 adjusts the power supplied to the heater 72 by changing the voltage applied to the heater 72 based on a control signal from the control unit 96 when energizing the heater 72.

In addition, the control device 95, which includes the variable power sources 24, 46, 52, and the heater power source 76 shown in FIG. 2, is actually connected to the electrodes inside the sensor element 101 through unillustrated lead wires formed within the sensor element 101 and unillustrated connector electrodes formed at the rear end of the sensor element 101 (only the heater connector electrode 71 shown in FIG. 1).

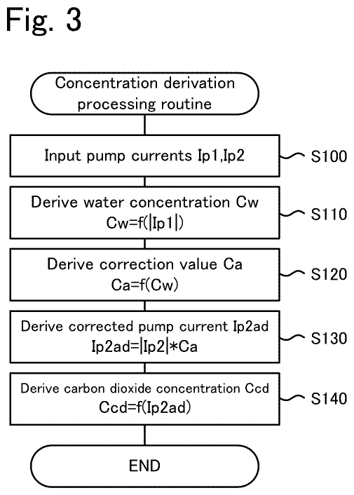

Next, an example of the processing by which the control unit 96 of the gas sensor 100 derives the concentrations of the specific gases (in this case, water concentration and carbon dioxide concentration) in the measurement gas will be described. The CPU 97 of the control unit 96 first performs the heater control processing described above to control the temperature of the heater 72 to reach a target temperature (e.g., 800° C.). Once the temperature of the heater 72 reaches (or approaches) the target temperature, the CPU 97 starts control of each of the pump cells 21, 41, 50 described above (i.e., the main pump control processing, first measurement pump control processing, and second measurement pump control processing) and acquires voltages V0, V1, V2, and Vref from the sensor cells 80 to 83 described above. Then, the CPU 97 derives the concentrations of the specific gases in the measurement gas based on the pump current Ip1 and the pump current Ip2. FIG. 3 is a flowchart showing an example of the concentration derivation processing routine performed by the CPU 97 of the control unit 96. The control unit 96 stores this routine in the storage unit 98, for example.

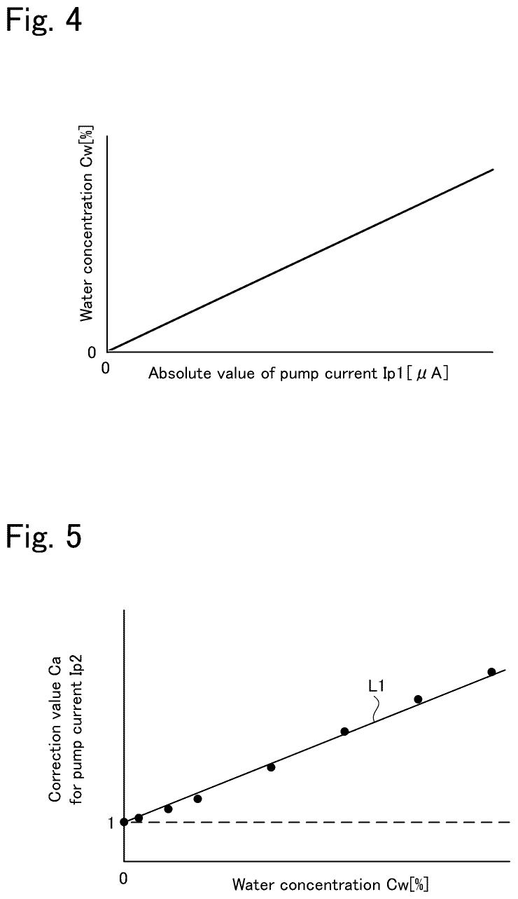

When the CPU 97 of the control unit 96 starts the concentration derivation processing routine, the CPU 97 first inputs the pump current Ip1 flowing through the first measurement pump cell 50 by the first measurement pump control processing, and the pump current Ip2 flowing through the second measurement pump cell 41 by the second measurement pump control processing (Step S100). Then, the CPU 97 derives the water concentration Cw in the measurement gas based on the input value of pump current Ip1 (the detected value of pump current Ip1) (Step S110). The CPU 97 derives the water concentration Cw, for example, using a water concentration derivation map. The water concentration derivation map is predetermined through experiments or analysis and so forth as a relationship between the absolute value of the pump current Ip1 and the water concentration Cw and stored in the storage unit 98. FIG. 4 is an explanatory diagram showing one example of the water concentration derivation map. As described above, the pump current Ip1 flowing through the first measurement pump cell 50 by the first measurement pump control processing correlates with the water concentration Cw in the measurement gas. For example, as shown in FIG. 4, there is a linear positive correlation between the absolute value of pump current Ip1 and water concentration Cw. In Step S110, the CPU 97 applies the absolute value of pump current Ip1 input in Step S100 to the correspondence relationship in FIG. 4, and derives the water concentration Cw corresponding to the absolute value of pump current Ip1. Thus, the water concentration in the measurement gas is measured.

Subsequently, the CPU 97 derives a correction value Ca based on the water concentration Cw (Step S120), and derives a corrected pump current Ip2ad by correcting the value of the pump current Ip2 (the detected value of the pump current Ip2) input in Step S100 using this correction value Ca (Step S130). In the present embodiment, the correction value Ca is a multiplication coefficient applied to the absolute value of the pump current Ip2, and the CPU 97 derives the corrected pump current Ip2ad by multiplying the absolute value of the pump current Ip2 by the correction value Ca. The derivation of the correction value Ca in Step S120 is performed, for example, using a correction value derivation map. The correction value derivation map is predetermined through experiments or analysis and so forth as a relationship between the water concentration Cw and the correction value Ca and stored in the storage unit 98. FIG. 5 is an explanatory diagram showing one example of the correction value derivation map. As shown by the straight line L1 in FIG. 5, there is a linear positive correlation between the water concentration Cw and the correction value Ca, and the higher the water concentration Cw, the greater the correction value Ca is set to be. In addition, the correction value Ca corresponding to the case where water concentration Cw of 0% is set to a value of 1, and in the case of the water concentration Cw of 0%, the absolute value of the pump current Ip2 is equal to the corrected pump current Ip2ad. In Step S120, the CPU 97 applies the water concentration Cw derived in Step S110 to the correspondence relationship shown by the straight line L1 in FIG. 5 and derives the correction value Ca corresponding to the water concentration Cw. Therefore, in Step S130, the correction is performed such that the higher the water concentration Cw, the more the absolute value of the pump current Ip2 tends to increase, and the corrected pump current Ip2ad is derived. The black circles in FIG. 5 will be described later.

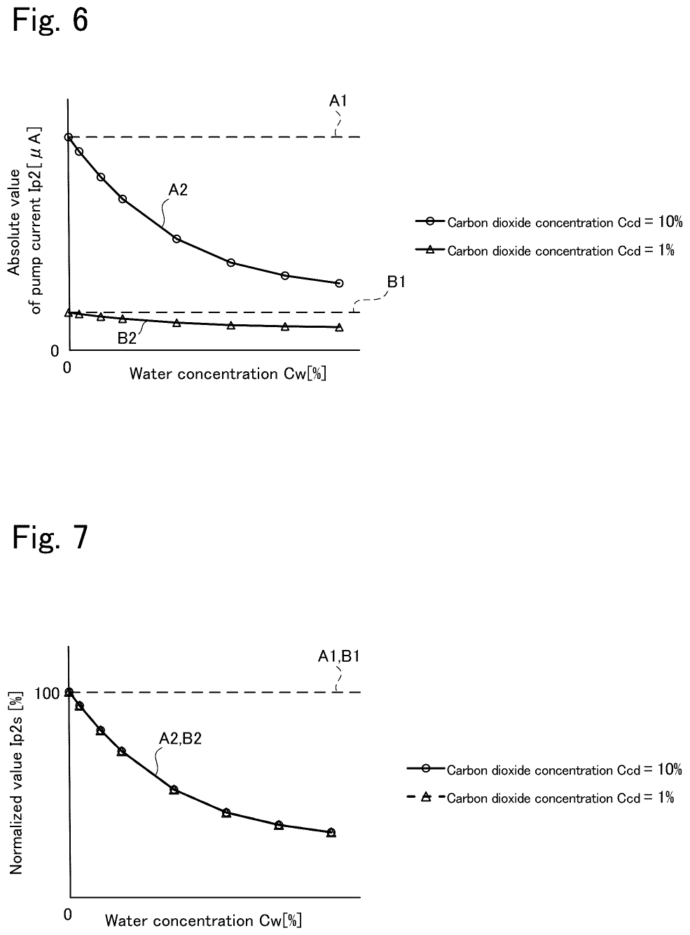

The reason for deriving the corrected pump current Ip2ad by correcting the absolute value of the pump current Ip2 using the correction value Ca derived from the correspondence relationship shown in FIG. 5 will be described. FIG. 6 is a graph showing the relationship between the water concentration Cw [%] and the absolute value of the pump current Ip2 [μA] when the water concentration Cw is varied with the carbon dioxide concentration Ccd in the measurement gas constant at a constant value. In FIG. 6, a straight line A1 represents an ideal graph in the case where the carbon dioxide concentration Ccd is constant at 10%, and a line graph A2 represents a line graph based on actual measurement values. Similarly, a straight line B1 represents an ideal graph in the case where the carbon dioxide concentration Ccd is constant at 1%, and a line graph B2 represents a line graph based on actual measurement values. As described above, since hydrogen derived from water in the measurement gas hardly reaches the third internal cavity 61, the pump current Ip2 correlates with the carbon dioxide concentration Ccd, and ideally, the pump current Ip2 should not change depending on the water concentration Cw. Therefore, ideally, as shown by the straight lines A1 and B1, if the carbon dioxide concentration Ccd is constant, the absolute value of the pump current Ip2 should remain constant regardless of the water concentration Cw. However, in practice, both line graphs A2 and B2 show that even when the carbon dioxide concentration Ccd is constant, the absolute value of the pump current Ip2 varies depending on the water concentration Cw. Specifically, it has been confirmed that the higher the water concentration Cw, the smaller the absolute value of the pump current Ip2 tends to become. In other words, it has been confirmed that the deviation between the straight lines A1 and B1 and the actual absolute value of the pump current Ip2 tends to increase as the water concentration Cw increases. The reason for this is considered to be that when the water concentration Cw in the measurement gas is higher than the carbon dioxide concentration Ccd, the reduction of water in the first internal cavity 20 by the main pump cell control processing becomes dominant, and the reduction of carbon dioxide in the first internal cavity 20 becomes insufficient. As a result, even when the carbon dioxide concentration Ccd is the same, the amount of carbon monoxide reaching the third internal cavity 61 (carbon monoxide derived from the carbon dioxide in the measurement gas) decreases as the water concentration Cw increases, and thus the absolute value of the pump current Ip2 flowing by the second measurement pump control processing is also considered to decrease. In this manner, since the absolute value of the pump current Ip2 decreases depending on the water concentration Cw, there is a concern that simply deriving the carbon dioxide concentration Ccd from the absolute value of the pump current Ip2 would result in a decrease in the measurement accuracy of the carbon dioxide concentration Ccd. For this reason, in the present embodiment, as described above, the absolute value of the pump current Ip2 is corrected using the correction value Ca derived based on the water concentration Cw, and the corrected pump current Ip2ad is derived.

FIG. 7 is a graph showing the relationship between the water concentration Cw [%] and the normalized value Ip2s [%], in a case where the ideal value of the absolute value of the pump current Ip2 is set to 100%, and the absolute values of the pump current Ip2 in each graph of FIG. 6 are normalized to obtain the normalized value Ip2s [%]. As shown in FIG. 7, the line graphs A2 and B2 became almost the same after normalization. That is, in both cases where the carbon dioxide concentration Ccd is 10% and 1%, the relationship between the water concentration Cw [%] and the absolute value [%] of the pump current Ip2 relative to the ideal value remained almost the same. From this result, it can be understood that if the correction value Ca is defined as a multiplication coefficient corresponding to the water concentration Cw so as to correct the absolute value [%] of the pump current Ip2, which has decreased due to the influence of the water concentration Cw, restore (correct) to the ideal value (100%), the absolute value of the pump current Ip2 can be corrected with relatively high accuracy using the same correction value Ca, regardless of the magnitude of the carbon dioxide concentration Ccd (that is, the magnitude of the absolute value of the pump current Ip2). For example, if the absolute value [%] of the pump current Ip2 corresponding to a certain water concentration Cw is 25%, then the correction value Ca may be set to 4 (=100%/25%), regardless of the magnitude of the absolute value [A] of the pump current Ip2. Based on this result, the eight correction values Ca calculated from the eight data points shown in FIG. 7 are plotted as black circles in FIG. 5. The straight line L1 in FIG. 5, i.e., the correction value derivation map, was derived as an approximate line based on these eight plotted points. In the present embodiment, since the correction value Ca is derived in Step S120 using the correction value derivation map thus obtained, and the correction in Step S130 is performed using this correction value Ca, the influence of the water concentration Cw on the absolute value of the pump current Ip2 can be reduced, and the corrected pump current Ip2ad can be derived as the absolute value of the pump current Ip2 that more accurately corresponds to the carbon dioxide concentration Ccd. Furthermore, by using the correction value Ca as a multiplication coefficient for the absolute value of the pump current Ip2, it is possible to derive the corrected pump current Ip2ad by correcting the absolute value of the pump current Ip2 with relatively high accuracy using the same correction value Ca, regardless of the magnitude of the carbon dioxide concentration Ccd (regardless of the magnitude of the absolute value of the pump current Ip2).

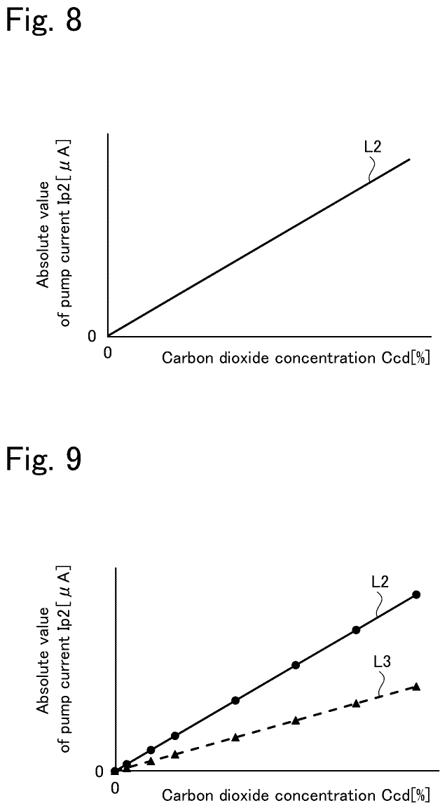

When the corrected pump current Ip2ad is derived in Step S130, the CPU 97 derives the carbon dioxide concentration Ccd in the measurement gas based on the corrected pump current Ip2ad (Step S140), and then ends the routine. In Step S140, the CPU 97 derives the carbon dioxide concentration Ccd using, for example, a carbon dioxide concentration derivation map. The carbon dioxide concentration derivation map is predetermined through experiments or analysis and so forth as a relationship between the absolute value of the pump current Ip2 and the carbon dioxide concentration Ccd and stored in the storage unit 98. FIG. 8 is an explanatory diagram showing one example of the carbon dioxide concentration derivation map. As described above, the pump current Ip2 flowing through the second measurement pump cell 41 by the second measurement pump control processing correlates with the carbon dioxide concentration Ccd in the measurement gas, and, for example, as shown by the straight line L2 in FIG. 8, there is a linear positive correlation between the absolute value of the pump current Ip2 and the carbon dioxide concentration Ccd. However, this straight line L2 represents the correspondence relationship between the absolute value of the pump current Ip2 and the carbon dioxide concentration Ccd in the case where the water concentration Cw in the measurement gas is 0%, that is, a graph obtained by investigating the ideal correspondence relationship between the absolute value of the pump current Ip2 and the carbon dioxide concentration Ccd. In Step S140, the CPU 97 applies the corrected pump current Ip2ad derived in Step S130 to the correspondence relationship shown by the straight line L2 in FIG. 8, in other words, uses the value of the corrected pump current Ip2ad as the absolute value of the pump current Ip2, and derives the carbon dioxide concentration Ccd corresponding to this value. In this manner, by deriving the carbon dioxide concentration Ccd using the corrected pump current Ip2ad, instead of the absolute value of the pump current Ip2 input in Step S100, it is possible to suppress a decrease in the measurement accuracy of carbon dioxide concentration Ccd caused by the water in the measurement gas.

A description will be given of the difference in measurement accuracy of the carbon dioxide concentration Ccd between a case where the absolute value of the pump current Ip2 input in Step S100 is used without correction, and a case where the corrected pump current Ip2ad is used. FIG. 9 is a graph showing the relationship between the carbon dioxide concentration Ccd [%] and the absolute value of the pump current Ip2 [μA]. In FIG. 9, the straight line L2 from FIG. 8 is also shown. The eight triangular data points in FIG. 9 plot the relationship between the carbon dioxide concentration Ccd (not the value derived by the gas sensor 100 but the true value of the carbon dioxide concentration Ccd in the model gas) and the absolute value of the pump current Ip2 input in Step S100, under conditions where the water concentration Cw has a nonzero value (e.g., 10%), as investigated using a model gas. In this way, when the measurement gas contains water, the correspondence relationship between the uncorrected absolute value of the pump current Ip2 and the true carbon dioxide concentration Ccd becomes like a line L3 that is deviated from the line L2. This deviation is the same as the deviation shown in FIGS. 6 and 7 between the straight lines A1, B1 and the line graphs A2, B2 (i.e., decrease in the absolute value of the pump current Ip2). Due to this deviation, if the absolute value of the pump current Ip2 input in Step S100 is applied to the correspondence relationship defined by straight line L2 to derive the carbon dioxide concentration Ccd, the derived carbon dioxide concentration Ccd tends to be smaller than the true carbon dioxide concentration Ccd, resulting in decreased measurement accuracy. In contrast, data corrected by multiplying the absolute values of the pump current Ip2 of the eight points of data shown by triangles in FIG. 9 by the above-mentioned correction value Ca is shown by black circles in FIG. 9. As shown, the black circles lie almost on the same straight line as the straight line L2. From this result, it has been confirmed that the correspondence relationship between the corrected absolute value of the pump current Ip2, that is, the corrected pump current Ip2ad, and the true carbon dioxide concentration Ccd is nearly the same as the straight line L2. Therefore, by applying the corrected pump current Ip2ad to the correspondence relationship defined by the straight line L2 to derive the carbon dioxide concentration Ccd, the derived carbon dioxide concentration Ccd becomes almost equal to the true carbon dioxide concentration Ccd, and it has been confirmed that the carbon dioxide concentration Ccd can be measured with high accuracy.

In the gas sensor 100 of the present embodiment, if carbon monoxide derived from carbon dioxide is oxidized in the second internal cavity 40, the pump current Ip1 would vary not only with the water concentration Cw but also with the carbon dioxide concentration Ccd, raising a concern that the correlation between the absolute value of the pump current Ip1 and the water concentration Cw, which serves as the basis for deriving the correction value Ca, may be disrupted. However, the higher the water concentration Cw is relative to the carbon dioxide concentration Ccd, the lower such concern becomes. In addition, as described above, the decrease in the measurement accuracy of the carbon dioxide concentration Ccd caused by water in the measurement gas tends to become more pronounced as the water concentration Cw becomes higher relative to the carbon dioxide concentration Ccd. Therefore, the gas sensor 100 of the present embodiment is particularly suitable for detecting the carbon dioxide concentration Ccd in cases where the water concentration Cw in the measurement gas is higher than the carbon dioxide concentration Ccd, especially in cases where the water concentration Cw is sufficiently higher such that almost no oxidation of carbon monoxide derived from carbon dioxide occurs in the second internal cavity 40.

Here, the correspondence relationship between the elements according to the present embodiment and the elements according to the present invention will be clarified. The sensor element 101 according to the present embodiment corresponds to the sensor element according to the invention. The element body 102 corresponds to the element body. The first internal cavity 20 corresponds to the first chamber. The inner pump electrode 22 corresponds to the first inner electrode. The main pump cell 21 corresponds to the first pump cell. The second internal cavity 40 corresponds to the second chamber. The first measurement electrode 51 corresponds to the second inner electrode.

The first measurement pump cell 50 corresponds to the second pump cell. The third internal cavity 61 corresponds to the third chamber. The second measurement electrode 44 corresponds to the third inner electrode. The second measurement pump cell 41 corresponds to the third pump cell. The outer pump electrode 23 corresponds to the first outer electrode, the second outer electrode, and the third outer electrode. The control device 95 corresponds to the control device. The main pump control processing corresponds to the first pump cell control processing. The first measurement pump control processing corresponds to the second pump cell control processing. The second measurement pump control processing corresponds to the third pump cell control processing. The pump current Ip2 corresponds to the third pump current. The pump current Ip1 corresponds to the second pump current. The absolute value of the pump current Ip1 corresponds to the absolute value of the second pump current. The absolute value of the pump current Ip2 corresponds to the absolute value of the third pump current. The corrected pump current Ip2ad corresponds to the corrected third pump current. The correction value Ca corresponds to the correction value. The reference electrode 42 corresponds to the reference electrode. The voltage V0 corresponds to the first voltage. The target value V0* corresponds to the first voltage target value. The voltage V1 corresponds to the second voltage. The target value V1* corresponds to the second voltage target value. The voltage V2 corresponds to the third voltage. The target value V2* corresponds to the third voltage target value.

According to the gas sensor 100 of the present embodiment described in detail above, the control device 95 derives the carbon dioxide concentration Ccd in the measurement gas based on the pump current Ip2, while taking into account the pump current Ip1. More specifically, the control device 95 derives the corrected pump current Ip2ad by correcting the pump current Ip2 taking into account the pump current Ip1, and then derives the carbon dioxide concentration Ccd based on the derived corrected pump current Ip2ad. Still more specifically, the control device 95 derives the water concentration Cw based on the absolute value of the pump current Ip1, derives the correction value Ca based on the derived water concentration Cw, and corrects the pump current Ip2 using the derived correction value Ca, thereby deriving the corrected pump current Ip2ad. Then, the control device 95 derives the carbon dioxide concentration Ccd based on the corrected pump current Ip2ad. As a result, the decrease in the measurement accuracy of the carbon dioxide concentration Ccd caused by the water in the measurement gas can be suppressed.

In addition, the control device 95 derives the corrected pump current Ip2ad by applying a correction in such a manner that the greater the absolute value of the pump current Ip1, the more the absolute value of the pump current Ip2 tends to increase. More specifically, the control device 95 derives a larger water concentration Cw as the absolute value of the pump current Ip1 increases, and applies a correction such that the greater the derived water concentration Cw, the more the absolute value of the pump current Ip2 tends to increase, thereby deriving the corrected pump current Ip2ad. By performing a correction according to such a tendency, it becomes possible to more reliably suppress a decrease in the measurement accuracy of the carbon dioxide concentration Ccd caused by the water in the measurement gas.

Furthermore, the correction value Ca is a multiplication coefficient for the absolute value of the pump current Ip2, and the control device 95 corrects the pump current Ip2 by multiplying the absolute value of the pump current Ip2 by the correction value Ca. The control device 95 corrects the pump current Ip2 using the same correction value Ca derived based on the absolute value of the pump current Ip1, regardless of the magnitude of the absolute value of the pump current Ip2. In this way, when the correction value Ca is the multiplication coefficient, relatively accurate correction of the pump current Ip2 can be achieved even using the same correction value Ca based on the absolute value of the pump current Ip1, regardless of the magnitude of the absolute value of the pump current Ip2. Therefore, the correction can be performed more easily compared to the case where the correction value Ca is derived based on not only the absolute value of the pump current Ip1 but also the absolute value of the pump current Ip2.

Furthermore, since the control device 95 also derives the water concentration Cw in the measurement gas based on the pump current Ip1, the gas sensor 100 can measure not only the carbon dioxide concentration Ccd but also the water concentration Cw.

It should be noted that the present invention is not limited to the present embodiment described above in any way, and it goes without saying that the present invention can be implemented in various modes as long as they fall within the technical scope of the present invention.

For example, in the above-described embodiment, the control device 95 derives the water concentration Cw based on the pump current Ip1, but the derivation of the water concentration Cw may be omitted. As described above, since the absolute value of the pump current Ip1 correlates with the water concentration Cw, it is possible to derive the carbon dioxide concentration Ccd by performing correction on the pump current Ip2 using the absolute value of the pump current Ip1 instead of the water concentration Cw, in the same manner as in the above-described embodiment. For example, the control device 95 may omit Step S110 in FIG. 3 and, instead of Step S120, derive the correction value Ca based on the absolute value of the pump current Ip1. In this case, instead of the correction value derivation map shown in FIG. 5, a correction value derivation map that defines, in advance by experiments or analysis and so forth, the correspondence relationship between the absolute value of the pump current Ip1 and the correction value Ca may be used. This correction value derivation map may also be defined, for example, based on FIGS. 4 and 5.

In the above-described embodiment, the correction value Ca is defined as a multiplication coefficient for the absolute value of the pump current Ip2, but the present invention is not limited thereto. For example, the correction value Ca may be an additive value applied to the absolute value of the pump current Ip2. In this case, as can be seen from the comparison between the line graphs A2 and B2 in FIG. 6, the correction value to be added in order to restore (correct) the absolute value of the pump current Ip2 to the ideal value varies not only with the absolute value of the pump current Ip1 (or the water concentration Cw based thereon), but also with the carbon dioxide concentration Ccd, i.e., the value of the absolute value of the pump current Ip2. Therefore, in a case where the correction value Ca is an additive value applied to the absolute value of the pump current Ip2, it is preferable to use a correction value derivation map, which defines, in advance by experiments or analysis and so forth, the correspondence relationship among the absolute value of the pump current Ip1 (or the water concentration Cw based thereon), the absolute value of the pump current Ip2, and the correction value Ca, instead of the correction value derivation map shown in FIG. 5. Even in a case where the correction value Ca is a multiplication coefficient applied to the absolute value of the pump current Ip2, as in the above-described embodiment, it is also acceptable to use a correction value derivation map that defines the correspondence relationship among the absolute value of the pump current Ip1 (or the water concentration Cw based thereon), the absolute value of the pump current Ip2, and the correction value Ca.

In the above-described embodiment, as shown by the straight line L1 in FIG. 5, the correction value derivation map is a map that represent a linear correspondence relationship between the water concentration Cw and the correction value Ca, however, the present invention is not limited thereto. For example, the correction value derivation map may be configured to perform correction such that the greater the absolute value of the pump current Ip1 (or the water concentration Cw based thereon), the more the absolute value of the pump current Ip2 tends to increase, and the correspondence relationship between the water concentration Cw and the correction value Ca may be a curved correspondence relationship or a step-function correspondence relationship. In addition, instead of the map as shown in FIG. 5, a relational expression (mathematical expression) representing the correspondence relationship between the absolute value of the pump current Ip1 (or the water concentration Cw based thereon) and the correction value Ca may be used. Similarly, the water concentration derivation map in FIG. 4 and the carbon dioxide concentration derivation map in FIG. 8 may also represent a curved or step-function correspondence relationship, and a relational expression (or mathematical expression) may be used instead of the map. Moreover, the correction value derivation map is not limited to one that applies the correction such that the greater the absolute value of the pump current Ip1 (or the water concentration Cw based thereon), the more the absolute value of the pump current Ip2 tends to increase. For example, depending on conditions, there may be cases in which the absolute value of the pump current Ip2 increases as the absolute value of the pump current Ip1 (or the water concentration Cw based thereon) increases. In such cases where the sensor element 101 is used under such conditions, it is also acceptable to use a correction value derivation map that performs correction such that the greater the absolute value of the pump current Ip1 (or the water concentration Cw based thereon), the more the absolute value of the pump current Ip2 tends to decrease.

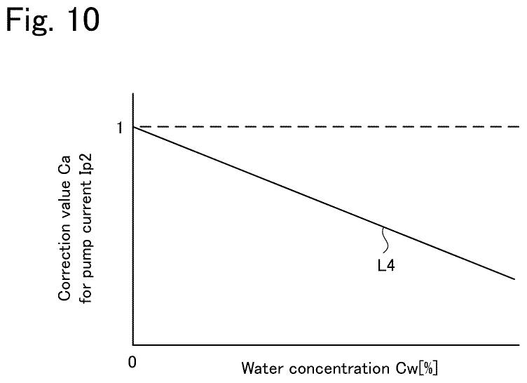

An example of such a correction value derivation map that performs correction such that the greater the absolute value of the pump current Ip1 (or the water concentration Cw based thereon), the more the absolute value of the pump current Ip2 tends to decrease is shown in FIG. 10. The correction value Ca in the correction value derivation map of FIG. 10 is, similarly to the correction value Ca in FIG. 5, a multiplication coefficient applied to the absolute value of the pump current Ip2. In the correction value derivation map shown in FIG. 10, as indicated by the straight line L4, there is a linear negative correlation between the water concentration Cw and the correction value Ca, and the correction value Ca is set to decrease as the water concentration Cw increases. In addition, the correction value Ca corresponding to a water concentration Cw of 0% is set to a value of 1, and when the water concentration Cw is 0%, the absolute value of the pump current Ip2 is equal to the corrected pump current Ip2ad. For example, in the gas sensor 100, if there is a tendency that the greater the absolute value of the pump current Ip1 (or the water concentration Cw based thereon), the greater the absolute value of the pump current Ip2 increases, the correction value derivation map shown in FIG. 10 may be stored in advance in the storage unit 98 instead of the correction value derivation map shown in FIG. 5. In this case, in Step S120 of the concentration derivation processing routine shown in FIG. 3, the CPU 97 applies the water concentration Cw derived in Step S110 to the correspondence relationship indicated by the straight line L4 in FIG. 10 to derive the correction value Ca corresponding to the water concentration Cw. Then, the CPU 97 derives the corrected pump current Ip2ad by performing correction using this correction value Ca (Step S130), and derives the carbon dioxide concentration Ccd based on the corrected pump current Ip2ad (Step S140). In this way, when the absolute value of the pump current Ip2 increases as the absolute value of the pump current Ip1 increases, the CPU 97 performs correction such that the greater the absolute value of the pump current Ip1, the more the absolute value of the pump current Ip2 tends to decrease, thereby it is possible to suppress a decrease in the measurement accuracy of the carbon dioxide concentration Ccd caused by water in the measurement gas.

The correction value derivation map shown in FIG. 10, like the correction value derivation map shown in FIG. 5, can be predetermined through experiments or analysis and so forth as the correspondence relationship between the water concentration Cw and the correction value Ca. For example, first, similar to the method used to obtain FIGS. 6 and 7 in the above-described embodiment, the relationship between the water concentration Cw [%] and the absolute value of the pump current Ip2 [μA] may be investigated while varying the water concentration Cw with the carbon dioxide concentration Ccd in the measurement gas held constant. Then, the correction value derivation map can be defined in such a manner that a correction value Ca is obtained to compensate for the deviation between the obtained relationship and the ideal relationship (restore the actual absolute value of the pump current Ip2 to its ideal val.

As a specific example of a case where the absolute value of the pump current Ip2 flowing by the second measurement pump control processing tends to increase as the water concentration Cw in the measurement gas increases, even when the carbon dioxide concentration Ccd in the measurement gas is the same, for example, it may be cited in a case when the amount of measurement gas reaching the first internal cavity 20 from the outside is relatively small, such as when the diffusion resistance from the outside of the element body 102 to the first internal cavity 20 in the measurement gas flow path is relatively large. For example, when the flow channel cross-sectional areas of the first diffusion rate-limiting section 11 and/or the second diffusion rate-limiting section 13 are relatively small, the diffusion resistance from outside the element body 102 to the first internal cavity 20 becomes relatively large, and the amount of measurement gas reaching the first internal cavity 20 from outside becomes relatively small. In such cases, the reason why the absolute value of the pump current Ip2 tends to increase as the water concentration Cw in the measurement gas increases can be considered as follows. First, when the amount of measurement gas reaching the first internal cavity 20 from outside the element body 102 is relatively small, the amount of carbon dioxide that needs to be reduced by the main pump control processing decreases, and therefore, the reduction of carbon dioxide in the first internal cavity 20 is less likely to be insufficient. On the other hand, since water (and hydrogen generated by the reduction of water) has a relatively high diffusion rate, water has an effect of promoting the diffusion of carbon dioxide (and carbon monoxide generated by the reduction of carbon dioxide) in the measurement gas. This is because molecular diffusion proceeds through collisions between molecules, and the diffusion rate (diffusion coefficient) of carbon dioxide (and carbon monoxide generated by the reduction of carbon dioxide) also changes depending on the diffusion rate of water (and hydrogen generated by the reduction of water). Therefore, the more water (and hydrogen generated by the reduction of water) is present in the measurement gas, the faster the diffusion of carbon dioxide (and carbon monoxide generated by the reduction of carbon dioxide) tends to be. Therefore, when the reduction of carbon dioxide in the first internal cavity 20 is less likely to become insufficient, the effect of water and hydrogen promoting the diffusion of carbon dioxide and carbon monoxide becomes dominant. As a result, it is considered that the higher the water concentration Cw in the measurement gas, the more the amount of carbon monoxide reaching the third internal cavity 61 increases, and a tendency appears in which the absolute value of the pump current Ip2 increases, even if the carbon dioxide concentration Ccd in the measurement gas is the same.

It goes without saying that the various aspects described in the above embodiment, the above modification, and the modification described later can also be applied to correction in cases where the absolute value of the pump current Ip2 tends to increase as the absolute value of the pump current Ip1 (or the water concentration Cw based thereon) increases. For example, although the correction value Ca in FIG. 10 is defined as a multiplication coefficient applied to the absolute value of the pump current Ip2, the correction value Ca may instead be defined as a subtraction value applied to the absolute value of the pump current Ip2. In such a case, a correction value derivation map different from that in FIG. 10 may be used. For example, a correction value derivation map that defines, in advance through experiments or analysis and so forth, the correspondence relationship among the absolute value of the pump current Ip1 (or the water concentration Cw based thereon), the absolute value of the pump current Ip2, and the correction value Ca may be used.

Furthermore, although the correction value derivation map in FIG. 10 represents a linear correspondence relationship between the water concentration Cw and the correction value Ca, the correspondence relationship may be curved correspondence relationship or step-function correspondence relationship. Alternatively, instead of using a map such as the one in FIG. 10, a relational expression (mathematical expression) may be used.

In the correction value derivation map shown in FIG. 10, the correction value Ca corresponding to the water concentration Cw of 0% was set to the value of 1, however, the present invention is not limited thereto. For example, a correction value derivation map may be used in which a correction value Ca of 1 is set for a water concentration Cw of an arbitrary value Cwa [%] (where Cwa is any value greater than 0% and equal to or less than 100%), and this value Cwa [%] is used as the basis for correction. In this case, instead of the carbon dioxide concentration derivation map shown in FIG. 8, a map based on the case where the water concentration Cw is Cwa [%] may be used. More specifically, a carbon dioxide concentration derivation map that defines in advance the correspondence relationship between the absolute value of the pump current Ip2 and the carbon dioxide concentration Ccd when the water concentration Cw is Cwa [%] (not 0%) may be stored in the storage unit 98. Then, in Step S130, the CPU 97 may derive the corrected pump current Ip2ad by using the correction value Ca derived from the correction value derivation map based on the value Cwa [%]. Further, in Step S140, the CPU 97 may derive the carbon dioxide concentration Ccd by applying the corrected pump current Ip2ad derived in Step S130 to the correspondence relationship shown by the carbon dioxide concentration derivation map based on the value Cwa [%]. The value Cwa may, for example, be the lowest value within the range in which the water concentration Cw in the measurement gas normally varies, or the value Cwa may be a most standard (frequent) value within the range in which the water concentration Cw in the measurement gas varies. The same applies to the correction value derivation map shown in FIG. 5.

In the above-described embodiment, the control device 95 derives a corrected pump current Ip2ad by performing a correction on the pump current Ip2, taking into account the water concentration Cw derived from the pump current Ip1, and derives the carbon dioxide concentration Ccd based on the corrected pump current Ip2ad. However, the present invention is not limited thereto. For example, the control device 95 may derive a provisional carbon dioxide concentration Ccdt based on the pump current Ip2, and then derive the carbon dioxide concentration Ccd by correcting the provisional carbon dioxide concentration Ccdt, taking into account the pump current Ip1. Even in this case, since the carbon dioxide concentration Ccd is derived based on the pump current Ip2 by taking into account the pump current Ip1, as in the above-described embodiment, it is possible to suppress a decrease in the measurement accuracy of the carbon dioxide concentration Ccd caused by water in the measurement gas. For example, when deriving the provisional carbon dioxide concentration Ccdt based on the pump current Ip2, the control device 95 may derive the provisional carbon dioxide concentration Ccdt as the carbon dioxide concentration obtained by applying the absolute value of the pump current Ip2 to the carbon dioxide concentration derivation map shown in FIG. 9. Further, when deriving the carbon dioxide concentration Ccd based on the provisional carbon dioxide concentration Ccdt by taking into account the absolute value of the pump current Ip1 (or the water concentration Cw based thereon), the carbon dioxide concentration Ccd may be derived by applying a correction in such a manner that the greater the absolute value of the pump current Ip1 (or the water concentration Cw based thereon), the more the provisional carbon dioxide concentration Ccdt tends to increased. In this case, for example, a correction value Cb may be derived based on the absolute value of the pump current Ip1 (or the water concentration Cw based thereon), and the provisional carbon dioxide concentration Ccdt may be corrected using the correction value Cb. The correction value Cb is used to correct the deviation between the provisional carbon dioxide concentration Ccdt and the carbon dioxide concentration Ccd caused by the water concentration Cw, and may be derived using the same map as the correction value derivation map for correction value Ca shown in FIG. 5, or a map that is different from FIG. 5 and represent the correspondence relationship between the absolute value of the pump current Ip1 (or the water concentration Cw based thereon) and the correction value Cb may be defined in advance, by experiments or analysis and so forth. The correction value Cb may be a multiplication coefficient applied to the provisional carbon dioxide concentration Ccdt. In this case, the provisional carbon dioxide concentration Ccdt may be multiplied by the correction value Cb to derive the corrected provisional carbon dioxide concentration Ccdt, that is, the carbon dioxide concentration Ccd. When the correction value Cb is used as the multiplication coefficient, the provisional carbon dioxide concentration Ccdt may be corrected using the same correction value Cb derived based on the absolute value of the pump current Ip1 (or the water concentration Cw based thereon), regardless of the magnitude of the provisional carbon dioxide concentration Ccdt. Alternatively, the correction value Cb may be derived using a correction value derivation map that defines, in advance by experiments or analysis and so forth, the correspondence relationship among the absolute value of the pump current Ip1 (or the water concentration Cw based thereon), the provisional carbon dioxide concentration Ccdt, and the correction value Cb. Furthermore, various modifications similar to those described for the correction value Ca may also be applied to the correction value Cb.

In the above-described embodiment, the water concentration Cw is derived based on the pump current Ip1, the correction value Ca is derived based on the derived water concentration Cw, the absolute value of the pump current Ip2 is corrected using the derived correction value Ca, thereby the corrected pump current Ip2ad is derived, and the carbon dioxide concentration Ccd is derived based on the corrected pump current Ip2ad. In the modification, the provisional carbon dioxide concentration Ccdt is derived based on the pump current Ip2, and then the carbon dioxide concentration Ccd was derived by correcting the provisional carbon dioxide concentration Ccdt based on the pump current Ip1. However, the present invention is not limited thereto, it is sufficient as long as the carbon dioxide concentration Ccd is derived based on the pump current Ip2, while taking into account the pump current Ip1. For example, the carbon dioxide concentration Ccd may be derived by applying both the pump current Ip2 and the pump current Ip1 to a predetermined correspondence relationship among the pump current Ip2, the pump current Ip1, and the carbon dioxide concentration Ccd. For example, this correspondence relationship can be defined based on FIGS. 4 to 8. If there is the relationship such that the greater the absolute value of the pump current Ip1 (or the water concentration Cw based thereon), the greater the absolute value of the pump current Ip2 as described above, then a correspondence relationship different from those in FIGS. 4 to 8 may be used.

In the above-described embodiment, the outer pump electrode 23 plays a role as the first outer electrode to be paired with the inner pump electrode 22 of the main pump cell 21, a role as the second outer electrode to be paired with the first measurement electrode 51 of the first measurement pump cell 50, and a role as the third outer electrode to be paired with the second measurement electrode 44 of the second measurement pump cell 41. In other words, the first to third outer electrodes are configured as a common outer pump electrode 23. However, the present invention is not limited thereto. For example, two of the first to third outer electrodes may be configured as a common electrode, and the remaining one may be provided outside the element body 102 as an electrode independent of the outer pump electrode 23 so as to be in contact with the measurement gas. Alternatively, the first to third outer electrodes may each be provided as independent electrodes outside the element body 102 so as to be in contact with the measurement gas.

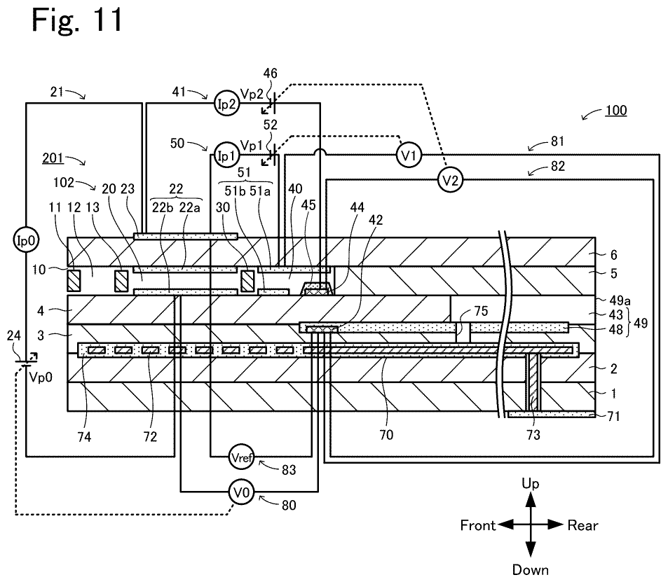

In the above-described embodiment, the sensor element 101 of the gas sensor 100 includes the first internal cavity 20, the second internal cavity 40, and the third internal cavity 61. However, the present invention is not limited thereto. For example, as shown in FIG. 11, a sensor element 201 according to a modification may not include the third internal cavity 61. In the sensor element 201 according to a modification shown in FIG. 11, between the lower surface of the second solid electrolyte layer 6 and the upper surface of the first solid electrolyte layer 4, the following components are adjacently formed and in communication with each other, in the order listed: a gas inlet 10; a first diffusion rate-limiting section 11; a buffer space 12; a second diffusion rate-limiting section 13; a first internal cavity 20; a third diffusion rate-limiting section 30; and a second internal cavity 40. In addition, the second measurement electrode 44 is disposed on the upper surface of the first solid electrolyte layer 4 within the second internal cavity 40. The second measurement electrode 44 is covered with a fourth diffusion rate-limiting section 45, which is a film made of a porous ceramic material such as alumina (Al₂O₃). The fourth diffusion rate-controlling section 45, similarly to the fourth diffusion rate-controlling section 60 of the above-described embodiment, serves to impart a predetermined diffusion resistance to the measurement gas in the second internal cavity 40 and guide the measurement gas to the second measurement electrode 44. Furthermore, the fourth diffusion rate-limiting section 45 also functions as a protective film for the measurement electrode 44. The ceiling electrode portion 51a of the first measurement electrode 51 extends directly above the second measurement electrode 44. Even with such a configuration of the sensor element 201, the carbon dioxide concentration Ccd can be detected based on the pump current Ip2 flowing through the second measurement pump cell 41, similarly to the embodiment described above. In the sensor element 201 of FIG. 11, the region around the second measurement electrode 44 functions as the third chamber. That is, the area around the second measurement electrode 44 serves the same role as the third internal cavity 61.

In the above-described embodiment, the element body 102 of the sensor element 101 is formed as the laminated body including multiple solid electrolyte layers (layers 1 to 6). However, the present invention is not limited thereto. The element body of the sensor element 101 only needs to include at least one oxygen ion-conductive solid electrolyte layer, and have a measurement gas flow path therein. For example, in FIG. 1, layers 1 to 5, except for the second solid electrolyte layer 6, may be structural layers made of materials other than solid electrolyte (e.g., layers made of alumina). In such a case, each electrode included in the sensor element 101 may be disposed on the second solid electrolyte layer 6. For instance, the second measurement electrode 44 shown in FIG. 1 may be disposed on a lower surface of the second solid electrolyte layer 6. Furthermore, the reference gas introduction space 43, which is formed in the first solid electrolyte layer 4, may instead be formed in the spacer layer 5. Likewise, the reference gas introduction layer 48, located between the first solid electrolyte layer 4 and the third substrate layer 3, may instead be provided between the second solid electrolyte layer 6 and the spacer layer 5. In addition, the reference electrode 42 may be provided on the lower surface of the second solid electrolyte layer 6 at a position downstream of the third internal cavity 61.

In the above-described embodiment, the first diffusion rate-limiting section 11, the second diffusion rate-limiting section 13, and the third diffusion rate-limiting section 30 are each provided as two horizontally elongated slits, but the present invention is not limited thereto. For example, one or more of the first diffusion rate-limiting section 11, the second diffusion rate-limiting section 13, and the third diffusion rate-limiting section 30 may be configured as a single horizontal elongated slit.

The present application is based upon and claims the benefit of priority from Japanese Patent Application No. 2023-055669, filed on Mar. 30, 2023, the entire contents of which are incorporated herein by reference. International Application No. PCT/JP2024/006281, filed on Feb. 21, 2024, is incorporated herein by reference in its entirety.

What is claimed is:

1. A gas sensor comprising a sensor element and a control device, and configured to measure a carbon dioxide concentration in a measurement gas,
wherein:
the sensor element includes:
an element body having an oxygen-ion-conductive solid electrolyte layer and a measurement gas flow path formed therein for introducing and allowing flow of the measurement gas;
a first pump cell including a first inner electrode disposed in a first chamber of the measurement gas flow path, and a first outer electrode disposed on the outside of the element body;
a second pump cell including a second inner electrode disposed in a second chamber located downstream of the first chamber of the measurement gas flow path, and a second outer electrode disposed on the outside of the element body;
a third pump cell including a third inner electrode disposed in a third chamber located downstream of the second chamber of the measurement gas flow path, and a third outer electrode disposed on the outside of the element body;
the control device performs:
a first pump cell control processing in which oxygen is pumped out from around the first inner electrode to around the first outer electrode by controlling the first pump cell, thereby reducing water and carbon dioxide in the measurement gas in the first chamber;
a second pump cell control processing in which oxygen is pumped from around the second outer electrode to around the second inner electrode by controlling the second pump cell, thereby oxidizing hydrogen generated by the reduction of water in the first chamber in the second chamber;
a third pump cell control processing in which oxygen is pumped from around the third outer electrode to around the third inner electrode by controlling the third pump cell, thereby oxidizing carbon monoxide generated by the reduction of carbon dioxide in the first chamber in the third chamber; and
the control device derives the carbon dioxide concentration in the measurement gas based on the third pump current flowing through the third pump cell by the third pump cell control processing, while taking into account the second pump current flowing through the second pump cell by the second pump cell control processing.

2. The gas sensor according to claim 1,
wherein the control device derives the carbon dioxide concentration based on a corrected third pump current obtained by correcting the third pump current taking into account the second pump current, or derives the carbon dioxide concentration by correcting a provisional carbon dioxide concentration based on the third pump current taking into account the second pump current.

3. The gas sensor according to claim 2,
wherein the control device derives the corrected third pump current or the carbon dioxide concentration by applying a correction in such a manner that the greater the absolute value of the second pump current, the more the absolute value of the third pump current or the provisional carbon dioxide concentration tends to increase.

4. The gas sensor according to claim 2,
wherein the control device derives the corrected third pump current or the carbon dioxide concentration by applying a correction in such a manner that the greater the absolute value of the second pump current, the more the absolute value of the third pump current or the provisional carbon dioxide concentration tends to decrease.

5. The gas sensor according to claim 2,
wherein the control device derives a correction value based on the absolute value of the second pump current, and corrects the third pump current or the provisional carbon dioxide concentration using the correction value.

6. The gas sensor according to claim 5,
wherein the correction value is a multiplication coefficient for the absolute value of the third pump current or the provisional carbon dioxide concentration, and
the control device corrects the third pump current or the provisional carbon dioxide concentration by multiplying the absolute value of the third pump current or the provisional carbon dioxide concentration by the correction value.

7. The gas sensor according to claim 6,
wherein the control device corrects the third pump current or the provisional carbon dioxide concentration using the same correction value derived based on the absolute value of the second pump current, regardless of the magnitude of the absolute value of the third pump current or the provisional carbon dioxide concentration.

8. The gas sensor according to claim 1,
wherein the control device derives a water concentration in the measurement gas based on the second pump current.

9. The gas sensor according to claim 1,
wherein at least two of the first outer electrode, the second outer electrode, and the third outer electrode are shared electrodes.

10. The gas sensor according to claim 1,
wherein the sensor element includes a reference electrode disposed inside the element body to contact a reference gas, and
the control device controls:
the first pump cell such that a first voltage between the reference electrode and the first inner electrode reaches a first voltage target value in the first pump cell control processing;
the second pump cell such that a second voltage between the reference electrode and the second inner electrode reaches a second voltage target value in the second pump cell control processing; and
the third pump cell such that a third voltage between the reference electrode and the third inner electrode reaches a third voltage target value in the third pump cell control processing.
